(19) United States
(12) Patent Application Publication
(10) Pub. No.: US 2005/0203537 A1
(43) Pub. Date: Sep. 15, 2005

(54) Orthopaedic injection restrictor apparatus and method

(76) Inventors: Roy C. Wiley, Warsaw, IN (US); Jayme M. Isham, Kalamazoo, MI (US); Bernard F. Morrey, Rochester, MN (US); Steven E. Stump, Goshen, IN (US)

Correspondence Address: ZIMMER TECHNOLOGY - ROBERTS, P.O. BOX 1268, ALEDO, TX, 76008, US

(21) Appl. No.: 10/798,217

(22) Filed: Mar. 9, 2004

(51) Int. Cl. A61B 17/58

(52) U.S. Cl. 606/93

(57) ABSTRACT

An orthopaedic injection restrictor apparatus is provided. The apparatus includes a nozzle. The nozzle includes an elongated portion. The apparatus further includes an orthopaedic plug. The orthopaedic plug includes a first portion releasably engaged with the elongated portion of the nozzle and further includes a plurality of flaps extending radially outwardly from its first portion. A method for restricting injection of a substance into a cavity within a bone with a plug is also provided. The method includes the steps of injecting the substance into the cavity and ejecting the plug into the cavity simultaneously with the injecting step.

### FIELD OF THE INVENTION

The present invention relates generally to the field of orthopaedics, and, more particularly, to an orthopaedic injection restrictor apparatus and method.

### BACKGROUND

Total joint arthroplasty (“joint replacement”) is the surgical replacement of a joint with a prosthesis. A typical conventional joint replacement procedure often includes anchoring a longitudinal stem, keel, post, or the like of the prosthesis into the medullary canal of a bone of the affected joint with an acrylic polymer or other suitable synthetic cement.

For some such procedures, it has been desirable to restrict the initial injection of the cement to a particular depth within the medullary canal.

### SUMMARY OF THE INVENTION

The present invention provides an orthopaedic injection restrictor apparatus. The apparatus includes a nozzle. The nozzle includes an elongated portion. The apparatus further includes an orthopaedic plug. The orthopaedic plug includes a first portion releasably engaged with the elongated portion of the nozzle and further includes a plurality of flaps extending radially outwardly from its first portion.

In an alternative embodiment, the present invention provides an apparatus for restricting injection of a substance to a depth in a cavity within a bone. The apparatus includes a means for injecting the substance into the cavity and a means, releasably engaged with the injecting means, for plugging the cavity.

In another alternative embodiment, the present invention provides a method for restricting injection of a substance into a cavity within a bone with a plug. The method includes the steps of injecting the substance into the cavity and ejecting the plug into the cavity simultaneously with the injecting step.

The above-noted features and advantages of the present invention, as well as additional features and advantages, will be readily apparent to those skilled in the art upon reference to the following detailed description and the accompanying drawings.

### BRIEF DESCRIPTION OF THE DRAWINGS

FIG. 1 shows a perspective view of an exemplary orthopaedic injection restrictor apparatus according to the present invention;

FIG. 2 shows a cross-sectional view of the exemplary injection nozzle of FIG. 1 (taken along line 2-2 of FIG. 1);

FIG. 3 shows a perspective view of the exemplary orthopaedic plug (predominantly from its nozzle engagement side) of FIG. 1;

FIG. 4 shows a plan view of the exemplary orthopaedic plug (from its nozzle engagement side) of FIG. 1 and FIG. 3;

FIG. 5 shows a cross-sectional view of the exemplary orthopaedic plug of FIG. 1, FIG. 3, and FIG. 4 (taken along line 5-5 of FIG. 4);

FIG. 6 shows a perspective view of an exemplary alternative embodiment of the orthopaedic plug (predominantly from its nozzle engagement side) of FIG. 1;

FIG. 7 shows a plan view of the exemplary alternative orthopaedic plug (from its nozzle engagement side) of FIG. 6; and

FIG. 8 shows a cross-sectional view of the exemplary alternative orthopaedic plug of FIG. 6 and FIG. 7 (taken along line 8-8 of FIG. 7).

### DETAILED DESCRIPTION OF EXEMPLARY EMBODIMENT(S)

Like reference numerals refer to like parts throughout the following description.

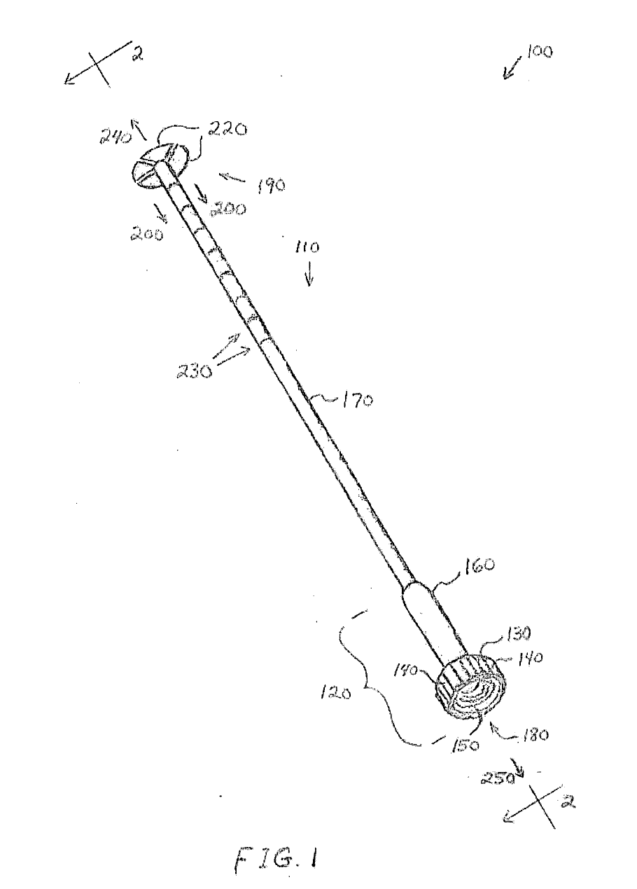

FIG. 1 shows a perspective view of an exemplary orthopaedic injection restrictor apparatus 100 according to the present invention. Exemplary apparatus 100 is of suitable size and weight for manipulation by hand and extension into an intramedullary canal, a pre-drilled bone site, or any other similar bone cavity such as those typically involved in a total or partial joint arthroplasty.

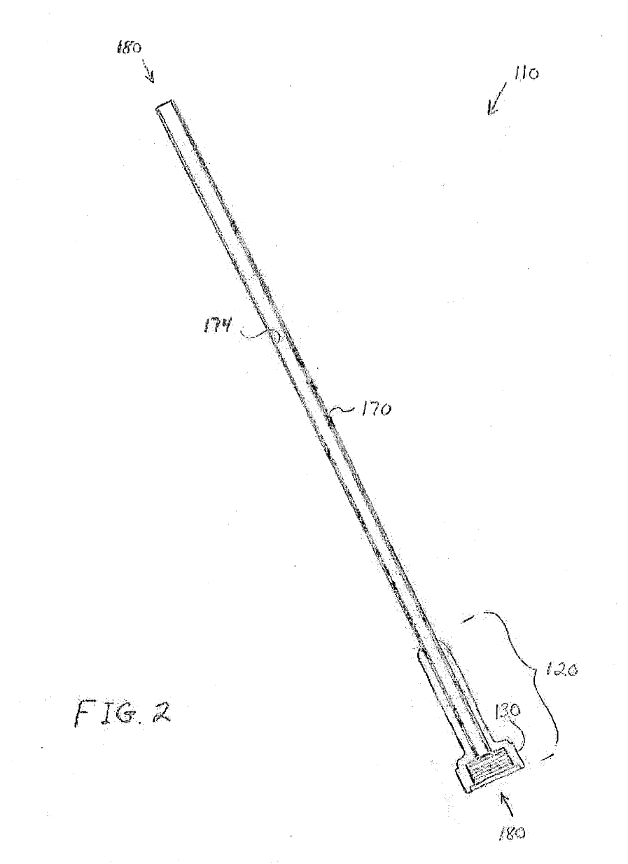

Exemplary apparatus 100 includes an exemplary injection nozzle 110. Among other things, nozzle 110 is configured to convey bone cement or any other suitable injection substance from a suitable controllable supply (not shown) into the bone cavity. In the exemplary embodiment nozzle 110 is disposable, made from injection molded High Density Polyethylene (“HDPE”), and gamma sterilized prior to use. In alternative embodiments, nozzle 110 may be reusable, made from any other suitable surgical material, and autoclaved or otherwise suitably sterilized prior to use. Nozzle 110 includes a base 120. Base 120 includes a generally cylindrical fitting 130. Among other things, fitting 130 is configured to connect to the injection substance supply. Fitting 130 has a plurality of generally planar external sidewall portions or flats 140 and internal screw threads 150. Base 120 also includes an elongated crown 160 extending axially from fitting 130. Nozzle 110 further includes an elongated tube 170 extending from crown 160. Tube 170 has a smooth inner sidewall 174 (see FIG. 2). Base 120 and tube 170 define a passageway or channel 180 having a generally T-shaped cross-section, with the shorter portion of the T defined by fitting 130 and the longer portion of the T defined by tube 170 (see FIG. 2). In the exemplary embodiment, tube 170 is flexible. In alternative embodiments, tube 170 may be rigid.

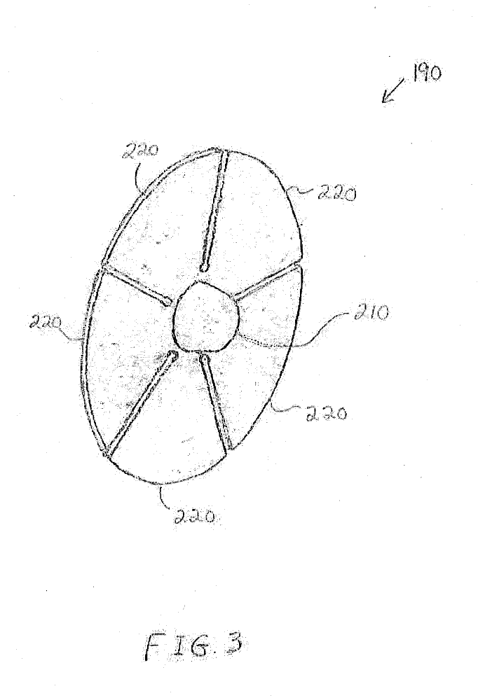

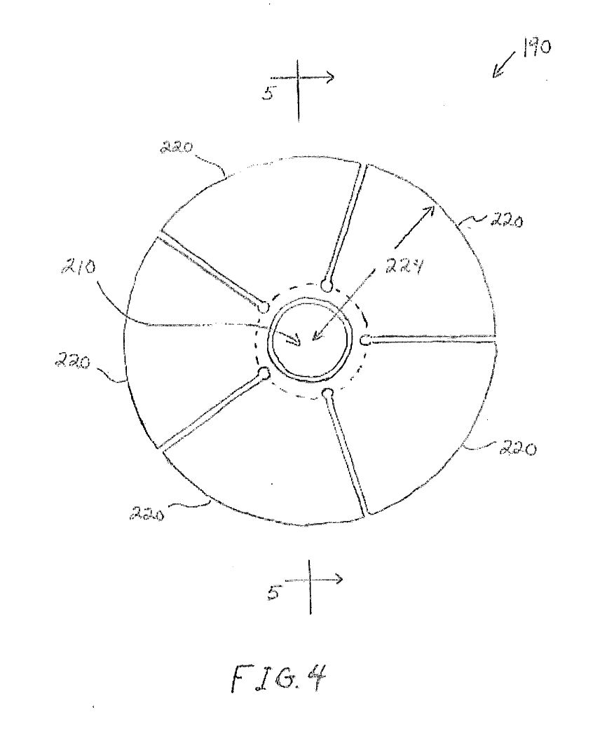

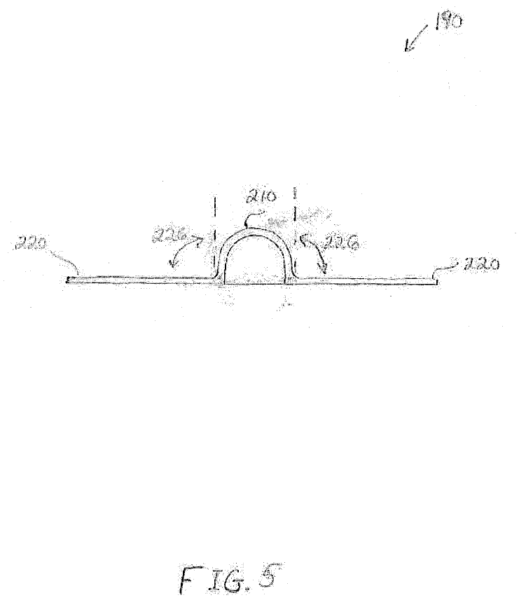

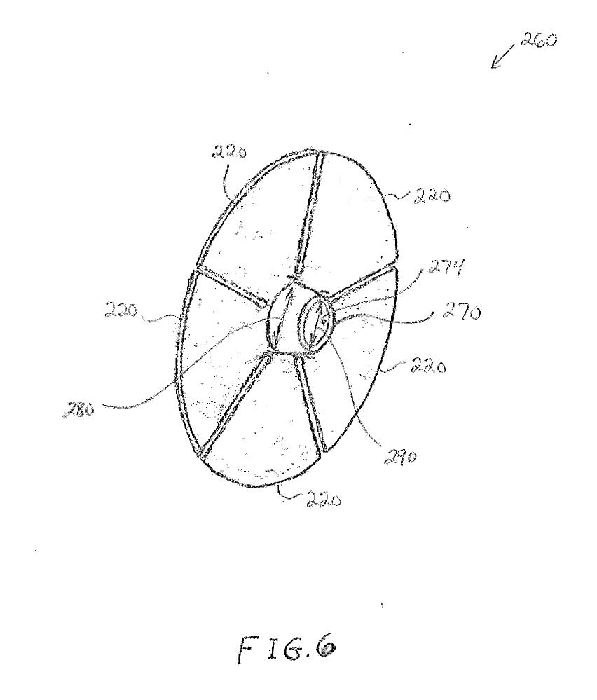

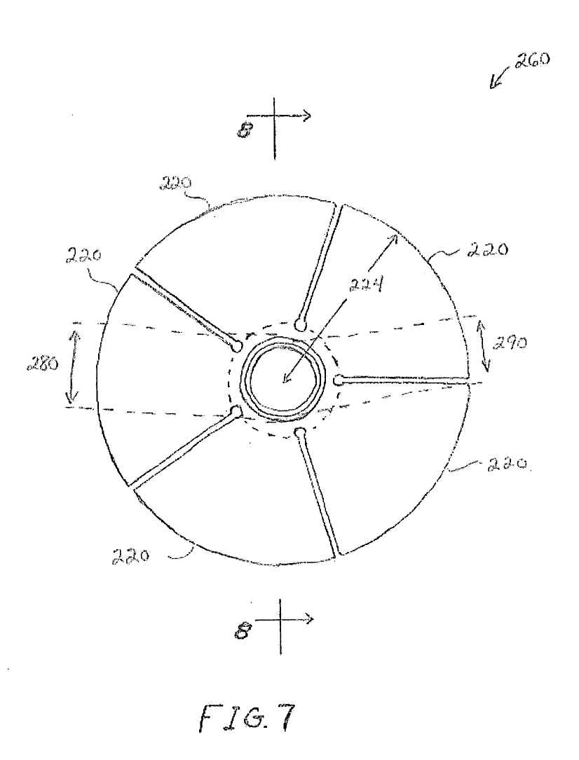

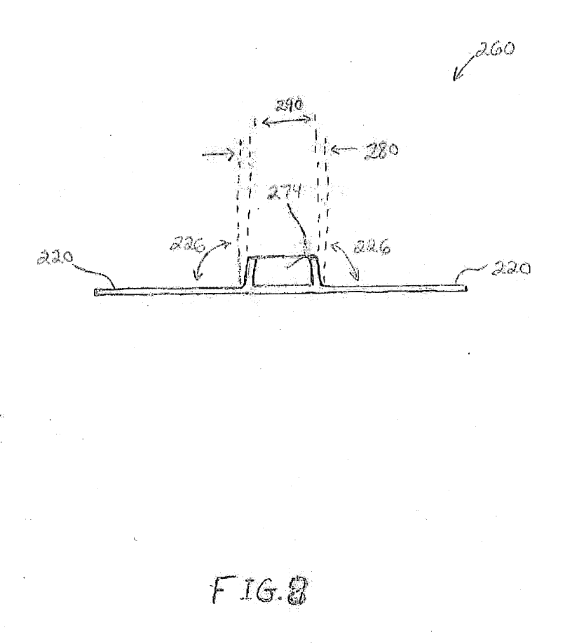

Exemplary apparatus 100 further includes an exemplary disk-like or fan-like orthopaedic plug 190. Among other things, plug 190 is configured to resiliently radially collapse or fold down to fit through a relatively smaller diameter opening leading into a relatively larger diameter cavity and to unfold or fan back out inside the cavity (after clearing the opening) to effectively plug or stopper the cavity vis-a-vis the injection substance. In the exemplary embodiment plug 190 is made from HDPE and gamma sterilized prior to use. In alternative embodiments, plug 190 may be made from any other suitable biocompatible material and autoclaved or otherwise suitably sterilized prior to use. In some of the alternative embodiments, plug 190 may also be bio-absorbable. Plug 190 has near side or a nozzle engagement side facing generally toward nozzle 110 as indicated by directional arrows 200. Plug 190 includes a centralized boss 210 (see FIG. 3, FIG. 4, and FIG. 5) and a plurality of flaps 220 extending radially outwardly from boss 210 (see also FIG. 3, FIG. 4, and FIG. 5). Boss 210 and tube 170 are sized to provide a suitable releasable pressed or interference fit between plug 190 and nozzle 110 wherein inner sidewall 174 of tube 170 sleeves around boss 210. In alternative embodiments, boss 210 may be replaced with a differently shaped stud, a ball, or any other structure suitable for providing a suitable fit between plug 190 and nozzle 110 so that injection of the substance suitably ejects plug 190 from nozzle 110 during operation as discussed further below (see, e.g. FIG. 6, FIG. 7, and FIG. 8 for one exemplary alternative).

In the exemplary embodiment the flexibility of the HDPE resiliently hinges flaps 220 to boss 210. Further, flaps 220 are sized to have a radius 224 (see FIG. 4) slightly greater than the radius of the cavity which is to be injected, such that when flaps 220 spread out against the inside of the cavity during operation they will rest against the cavity at an acute angle 226 relative to the longitude of tube 170 (see FIG. 1, FIG. 2 and FIG. 5). In alternative embodiments, suitable alternative resilient hinging between flaps 220 and boss 210 may be implemented with any other suitable alternative materials and/or structures.

Exemplary apparatus 100 further includes a plurality of graduations 230 marked on tube 170. In the exemplary embodiment, graduations 230 are implemented as black bands that are suitably painted or otherwise marked onto tube 170. In alternative embodiments, graduations 230 may be otherwise marked or embossed onto, etched or molded into, or affixed to tube 170 in any other suitable manner.

Directional arrows 240 and directional arrows 250 are discussed further below in connection with operation of exemplary apparatus 100.

FIG. 2 shows a cross-sectional view of exemplary injection nozzle 110 (taken along line 2-2 of FIG. 1). Base 120, tube 170, inner sidewall 174, and channel 180 are discernable in FIG. 2.

FIG. 3 shows a perspective view of exemplary orthopaedic plug 190 (predominantly from its nozzle engagement side). Boss 210 and flaps 220 are discernable in FIG. 3.

FIG. 4 shows a plan view of exemplary orthopaedic plug 190 (from its nozzle engagement side). Boss 210, flaps 220, and radius 224 are discernable in FIG. 4.

FIG. 5 shows a cross-sectional view of exemplary orthopaedic plug 190 (taken along line 5-5 of FIG. 4). Boss 210, two of flaps 220, and angle 226 are discernable in FIG. 5.

FIG. 6 shows a perspective view of an exemplary alternative embodiment 260 of orthopaedic plug 190 (predominantly from its nozzle engagement side; see directional arrows 200 of FIG. 1). Like exemplary plug 190, exemplary alternative plug 260 includes flaps 220. Like exemplary plug 190, flaps 220 have a radius 224 (see FIG. 7) and an operational angle 226 relative to the longitude of tube 170 (see FIG. 1, FIG. 2 and FIG. 8). However, for alternative plug 260, boss 210 (see FIG. 3) is replaced with a generally frusto-conical, centralized protuberance 270 having an inner sidewall 274, a first external circumference 280 proximal to flaps 220, and a second external circumference 290 distal to flaps 220 (see also FIG. 7). For exemplary plug 260, circumference 280 and circumference 290 are sized to provide a suitable releasable pressed or interference fit between plug 260 and nozzle 110 (see FIG. 1) wherein inner sidewall 174 of tube 170 (see FIG. 2) sleeves around protuberance 270. On the other hand, in alternative embodiments circumference 280 and circumference 290 are sized to provide a suitable releasable pressed or interference fit between plug 260 and nozzle 10 (see FIG. 1) wherein inner sidewall 274 of protuberance 270 sleeves around tube 170. Additionally, it should be appreciated that in alternative embodiments protuberance 270 and tube 170 taper may be further configured to provide a suitable releasable taper connection between plug 260 and nozzle 110. Moreover, in alternative embodiments protuberance 270 may be replaced with any other structure suitable for providing a suitable fit between plug 260 and nozzle 110.

FIG. 7 shows a plan view of exemplary alternative plug 260. Flaps 220, radius 224, circumference 280, and circumference 290 are discernable in FIG. 7.

FIG. 8 shows a cross-sectional view of exemplary alternative plug 260 (taken along line 8-8 of FIG. 7). Two of flaps 220, angle 226, inner sidewall 274, circumference 280, and circumference 290 are discernable in FIG. 8.

In operation of exemplary apparatus 100, a surgeon or other operator decides the depth to which a synthetic cement or other substance is to be injected into an intramedullary canal, a pre-drilled bone site, or any other similar bone cavity such as those typically involved in a total or partial joint arthroplasty. The operator may choose to set the depth to which the substance will be injected by removing plug 190 from tube 170 and then cutting off tube 170 at or about one of graduations 230, thus reducing the overall length of nozzle 110 to a corresponding known value; or, alternatively, the operator may choose to visually gauge the depth by reference to graduations 230 during insertion of tube 170 into the cavity as discussed further below. In any event, via fitting 130 nozzle 110 is connected to a suitable controllable supply of cement or other suitable injection substance.

Next, with boss 210 of plug 190 inserted into channel 180 of tube 170, plug 190 and tube 170 are inserted into the cavity along the general direction of arrow 240 (see FIG. 1). Flaps 220 fold down as necessary during insertion (i.e., angle 226 becomes more acute) as compared to their pre-insertion position but then they fan out or spread out against the inside of the cavity. If the depth has been set by cutting tube 170, then the remaining portion of tube 170 is fully inserted into the cavity (until nozzle 110 stops at base 120); otherwise, if visual depth gauging is desired, tube 170 is inserted into the cavity to a desired depth as visually determined by the position of graduations 230.

Next, the substance is injected into the cavity. The injection simultaneously ejects boss 210 (and thus, plug 190) from tube 170. Nozzle 110 is withdrawn from the cavity, while plug 190 remains lodged within the cavity (via flaps 220) to block the injected substance from spreading into undesired areas.

Similar operations for alternative plug 260 as opposed to plug 190 and/or for other alternative embodiments of apparatus 100 should be readily appreciated.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. An orthopaedic injection restrictor apparatus, comprising:
a nozzle including an elongated portion; and
an orthopaedic plug including a first portion releasably engaged with the elongated portion of the nozzle and further including a plurality of flaps extending radially outwardly from said first portion.

2. The apparatus of claim 1, wherein at least one of the flaps is resiliently hinged to the first portion of the orthopaedic plug.

3. The apparatus of claim 1, wherein the elongated portion of the nozzle defines a sleeve and the first portion of the orthopaedic plug partially extends into the sleeve.

4. The apparatus of claim 3, wherein the first portion of the orthopaedic plug includes a boss positioned within the sleeve.

5. The apparatus of claim 1, further comprising a plurality of graduations on the elongated portion of the nozzle.

6. The apparatus of claim 5, wherein the plurality of graduations extends linearly away from at least one of the flaps.

7. The apparatus of claim 1, wherein the elongated portion of the nozzle includes a flexible portion.

8. The apparatus of claim 1, wherein the first portion of the orthopaedic plug defines a sleeve and the elongated portion of the nozzle extends into the sleeve.

9. The apparatus of claim 2, further comprising:
a plurality of graduations on the elongated portion of the nozzle;
wherein the elongated portion of the nozzle defines a sleeve, the first portion of the orthopaedic plug extends into the sleeve, the plurality of graduations extends linearly away from at least one of the flaps, and the elongated portion of the nozzle includes a flexible portion.

10. The apparatus of claim 9, wherein the first portion of the orthopaedic plug includes a boss positioned within the sleeve.

11. The apparatus of claim 2, further comprising:
a plurality of graduations on the elongated portion of the nozzle;
wherein the first portion of the orthopaedic plug defines a sleeve and the elongated portion of the nozzle extends into the sleeve, the plurality of graduations extends linearly away from at least one of the flaps, and the elongated portion of the nozzle includes a flexible portion.

12. An apparatus for restricting injection of a substance to a depth in a cavity within a bone, comprising:
means for injecting the substance into the cavity; and
means, releasably engaged with the injecting means, for plugging the cavity.

13. The apparatus of claim 12, wherein the injecting means and the plugging means form a means for ejecting the plugging means from the injecting means simultaneously with injecting the substance into the cavity.

14. The apparatus of claim 12, wherein the injecting means includes a means for setting the depth.

15. A method for restricting injection of a substance into a cavity within a bone with a plug, the method comprising the steps of:
injecting the substance into the cavity; and
ejecting the plug into the cavity simultaneously with the injecting step.
